(19) United States
(12) Patent Application Publication
(10) Pub. No.: US 2026/0306721 A1
(43) Pub. Date: Oct. 8, 2026

(54) SOLUBLE BIOMOLECULAR GLASS AND USE THEREOF

(71) Applicant: INSTITUTE OF PROCESS ENGINEERING, CHINESE ACADEMY OF SCIENCES, Beijing (CN)

(72) Inventors: Xuehai YAN, Beijing (CN); Chengqian YUAN, Beijing (CN); Wei FAN, Beijing (CN)

(21) Appl. No.: 19/480,541

(22) PCT Filed: May 5, 2023
(86) PCT No.: PCT/CN2023/092307

§ 371 Date: Oct. 31, 2025

(51) Int. Cl. A61L 31/16; A61K 31/198

(52) U.S. Cl. CPC A61L 31/16; A61K 31/198; A61L 2300/214

(57) ABSTRACT

The present disclosure provides a biomolecular glass and use thereof, where the biomolecular glass is obtained by volatilizing a solvent of a biomolecular raw material solution, the biomolecular raw material solution includes a biomolecular raw material, a regulator, and the solvent; and the biomolecular raw material includes a natural amino acid or a peptide, or an amino acid derivative or a peptide derivative, or a mixture of two or more thereof. The soluble biomolecular glass in the present disclosure can be processed into an implantable or non-implantable medical material and/or device, filling and stent material, electronic sensing material and/or device, transient electronic material and/or device, etc., and can be degraded by organisms or nature with pollution-free degradation products.

### TECHNICAL FIELD

The present disclosure relates to a soluble biomolecular glass, a preparation method and use thereof, and belongs to the technical field of a material and a preparation method thereof. The present disclosure discloses a class of soluble biomolecular glasses based on amino acids and peptides, and a method for preparing a glass by solvent evaporation of a biomolecular solution. Such soluble biomolecular glasses can be processed into an implantable or non-implantable medical material and/or device, filling and stent material, electronic sensing material and/or device, transient electronic material and/or device, and can be used in the fields of medical care, health monitoring, security encryption, information transfer, and storage.

### BACKGROUND

Glass is an amorphous solid known for its transparency, high hardness, and corrosion resistance, and is widely used. For example: 1) glass is an important building material widely used in a building facade, a roof, a partition wall, a door and a window, a stair handrail, a skylight, etc.; and 2) glass has excellent electrical properties and is widely used in an electronic device, such as a display screen, a touch screen, and an optical fiber transmission chip. Glass has particular physical, chemical, and mechanical properties. With the continuous advancement and innovation of science and technology, the application fields of glass continue to expand.

Glass has very stable chemical properties. Glass products are not biodegradable, and can exist in the natural environment for a long time, thereby imposing a heavy burden on an ecological environment. Biomolecules, particularly amino acid and peptide molecules, have good biocompatibility and biodegradability. At present, researchers have prepared a biodegradable glass from biomolecules.

At present, the preparation of a biomolecular glass based on traditional melt-quenching method has been disclosed. For example, a biodegradable glass based on an amino acid, a peptide and a derivative has been disclosed (CN113754556B); and a cyclopeptide glass and a cyclopeptide-containing pharmaceutical composition glass have been disclosed (CN114014908A). However, there are major limitations in preparing an amino acid/peptide glass using the traditional melt-quenching method. First, amino acid/peptide molecules have a high melting point and are susceptible to thermal decomposition before melting at a high temperature; second, the high-temperature melting and low-temperature quenching process is complex, and not environmentally friendly; further, the amino acid/peptide molecules are prone to oxidation under high-temperature conditions, thereby affecting the light transmittance and transparency of glass. Moreover, heating changes the structure or conformation of the amino acid/peptide molecules, and seriously reduces the bioactivity of the amino acid/peptide. Therefore, the melt-quenching method is limited in the preparation of the biomolecular glass.

At present, no method for preparing an amino acid/peptide glass, other than the “melt-quenching” method, has been disclosed. Surprisingly, the present disclosure discovers that the amino acid/peptide molecules can form a glass in an aqueous solution by adding a regulator and subsequently volatilizing a solvent. Such an amino acid/peptide biomolecular glass is water-soluble, and can be degraded by organisms or nature with completely green and environmentally friendly degradation products. Compared with the traditional melt-quenching method, the discovered method is simpler, milder, more controllable, and more environmentally friendly.

The present disclosure is completed based on this discovery. The soluble biomolecular glass and the biomolecular glass material and/or device developed based on the present disclosure are expected to be used in the preparation of an implantable or non-implantable medical material and/or device, filling and stent material, electronic sensing material and/or device, transient electronic material and/or device, and can be used in the fields such as medical care, health monitoring, security encryption, information transfer, and storage.

### SUMMARY

A primary objective of the present disclosure is to provide a soluble biomolecular glass and a preparation method thereof. Main raw materials include a natural amino acid or a peptide, or an amino acid derivative or a peptide derivative, or a mixture of two or more thereof, a regulator, and an aqueous solvent. Different from the disclosed “melt-quenching” method for preparing an amino acid/peptide glass (CN 113754556 B, CN114014908A), the present disclosure discloses a method for preparing a glass by volatilizing a solvent in an aqueous biomolecular solution by heating and/or pressurizing the aqueous biomolecular solution.

The soluble biomolecular glass in the present disclosure exhibits excellent biocompatibility, high light transmittance, good mechanical strength, and versatile processability. The soluble glass thus prepared can be processed into an implantable or non-implantable medical material and/or device, filling and stent material, electronic sensing material and/or device, transient electronic material and/or device, and can be degraded by organisms or an ecological environment with completely green and environmentally friendly degradation products.

Use of the soluble biomolecular glass in the present disclosure and the preparation method therefor find applications in diverse fields, such as medical care, health monitoring, security encryption, information transfer, and storage.

In the first aspect, a biomolecular glass is obtained by volatilizing a solvent in a biomolecular raw material solution, the biomolecular raw material solution including a biomolecular raw material, a regulator, and the solvent; the biomolecular raw material including a natural amino acid or a peptide, or an amino acid derivative or a peptide derivative, or a mixture of two or more thereof.

In the second aspect, for the soluble biomolecular glass, the biomolecular raw material is a natural amino acid or a peptide, or an amino acid derivative or a peptide derivative, or a mixture of two or more thereof:
specifically,
is a natural amino acid molecule in the form of formula (1), or an amino acid derivative, or a pharmaceutically acceptable salt thereof, or a mixture of any one or two or more thereof,

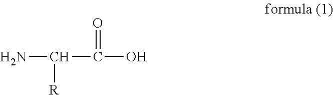

the amino acid includes glycine, alanine, valine, leucine, isoleucine, methionine (Met), proline, tryptophan, serine, tyrosine, cysteine, phenylalanine, asparagine, glutamine, threonine, aspartic acid, glutamic acid, lysine, arginine, histidine, selenocysteine, and pyrrolysine;
or is a peptide molecule formed by peptide bond condensation from formula (1), or a peptide derivative, or a pharmaceutically acceptable salt thereof, or a mixture of any one or two or more thereof.

The peptide is a molecule formed by peptide bond condensation of n of the natural amino acids, where n≥2; preferably, 2≤n≤10.

In the above amino acid or peptide derivative, the protecting group including but not limited to: Trt, Boc, Fmoc, Cbz/Z, Allyl, C2-C18 acyl, benzoyl, naphthoyl, OFm, Otbu, OBzl, OAll, OMe, and OEt.

In the third aspect, for the soluble biomolecular glass, the regulator is selected from the group consisting of a nucleotide, a nucleotide polymer, RNA, DNA and/or other regulator, or a mixture of two or more thereof,
for example,
the nucleotide includes, but is not limited to, adenosine-5′-monophosphate (AMP), nicotinamide monophosphate (NMN), guanosine-5′-monophosphate (GMP), uridine-5′-monophosphate (UMP), cytidine-5′-monophosphate (CMP), and adenosine-3′-monophosphate (3-AMP); and
the other regulator includes, but is not limited to, D/L-tartaric acid, hydrochloric acid, sulfuric acid, phosphoric acid, lactic acid, polylactic acid, hyaluronic acid, alginic acid, lactobionic acid, citric acid, tartaric acid, oxalic acid, D/L-malic acid, maleic acid, pseudolaric acid, acamprosate, quinic acid, adipic acid, fumaric acid, hexanoic acid, heptanoic acid, octanoic acid, valeric acid, butyric acid, propionic acid, and glacial acetic acid; preferably hydrochloric acid, nucleotide, citric acid, glacial acetic acid, and malic acid.

In the fourth aspect, for the soluble biomolecular glass, the solvent is water or a salt solution thereof.

In the fifth aspect, a method for preparing the soluble biomolecular glass includes the following steps:
(1) weighing a biomolecular raw material, uniformly mixing and grinding the biomolecular raw material;
(2) dissolving raw material powder from the step (1) in a solvent to obtain the biomolecular solution;
(3) adding a regulator to the biomolecular solution from the step (2), and adjusting a final pH value of the solution by controlling an amount of the regulator; where if the regulator is a mixed solution, it needs to be fully premixed; and
(4) transferring the aqueous solution from the step (3) in a container, and volatilizing the solvent by adjusting a temperature of the solution and/or pressurizing the solution, and/or vacuumizing the solution, to prepare the soluble biomolecular glass.

In the sixth aspect, for the soluble biomolecular glass and the preparation method thereof, after addition of the regulator, the biomolecular solution is at a pH of 1-8, preferably at a pH of 4-7.

In the seventh aspect, for the soluble biomolecular glass and the preparation method thereof, the solvent can be volatilized by air flow, spray, fluidized bed, spin flash, infrared, microwave, or freezing/vacuum freezing.

In the eighth aspect, for the soluble biomolecular glass and the preparation method thereof, the solvent volatilization time is 20 min to 7 h, preferably 40 min to 4 h.

In the ninth aspect, for the soluble biomolecular glass and the preparation method thereof, a mass percentage of the biomolecular raw material in the glass is 50 wt % to 99.5 wt %, preferably 85 wt % to 99 wt %; a mass percentage of the regulator in the glass is 0 to 50 wt %, preferably 0 to 15 wt %, further preferably greater than 0 wt %; and a mass percentage of the solvent in the glass is 0.05 wt % to 5 wt %, preferably 0.1 wt % to 2 wt %.

In the tenth aspect, for the soluble biomolecular glass and the preparation method thereof, a strong hydrogen-bond interaction exists between biomolecules and the regulator and/or the solvent, and the biomolecular glass is rich in hydrogen-bond networks.

In the eleventh aspect, the soluble biomolecular glass is an amorphous body in glassy state, that is, a non-crystalline solid, exhibiting isotropy, no constant melting point, and reversibility.

In the twelfth aspect, the soluble biomolecular glass has a glass transition temperature (Tg) adjustable within a range of 0 to 200° C.

Preferably, the Tg of the soluble biomolecular glass satisfies 20° C.<Tg<200° C.

More preferably, the Tg of the soluble biomolecular glass is in a range of 20° C.<Tg<160° C.

In the thirteenth aspect, the soluble biomolecular glass has a good glass-forming ability (GFA), that is, a ratio of the Tg (glass transition temperature)/Tm (melting temperature) is between 0.55 and 0.75, preferably 0.66-0.75.

In the fourteenth aspect, the soluble biomolecular glass has performance indicators as follows when measured at a thickness of 1 mm and a wavelength of 500 nm:
a light transmittance of 40% to 99%, preferably 80% to 99%; and
a hardness greater than 20 Mpa, preferably greater than 50 Mpa.

In the fifteenth aspect, the soluble biomolecular glass can be processed into an implantable or non-implantable medical material and/or device, filling and stent material, electronic sensing material and/or device, transient electronic material and/or device by the processing method of spin coating, casting, electrospinning, and 3D printing.

In the sixteenth aspect, the soluble biomolecular glass and the biomolecular glass material and/or device can be degraded by organisms or an ecological environment; preferably, can be degraded in a compost environment at room temperature and/or a human body temperature; and more preferably, can be transiently dissolved in an aqueous solution, blood, tissue fluid, and body fluid environment.

In the seventeenth aspect, the soluble biomolecular glass and the biomolecular glass material and/or device have dissolution time controllably adjusted in a range of 0.5 min to 3.5 months based on raw material, size, and structure of the glass; and adjustable and/or controllable dissolution rate based on the controllable adjustment of the raw material, the size, and the structure of the glass.

In the eighteenth aspect, the soluble biomolecular glass and the biomolecular glass material and/or device can be completely degraded and/or transiently dissolved.

In the nineteenth aspect, the soluble biomolecular glass and the biomolecular glass material and/or device can be used in fields, such as medical care, health monitoring, security encryption, information transfer, and storage:
preferably, can be used to process and prepare a soluble glass microneedle, which can be used to puncture skin and/or mucosa, blood-eye barrier, blood-brain barrier, etc., for pharmaceutical use;
and/or are used in a soluble supercapacitor, chip, glass electrode, transient circuit/memory, etc., for pharmaceutical or sensing or electronic use;
and/or are used in a soluble stent, preferably a bone stent, and/or a heart stent, and/or a vascular stent, etc., for pharmaceutical or medical use.

### BRIEF DESCRIPTION OF THE DRAWINGS

FIG. 1 is a photograph of an arginine glass prepared in Example 1.

FIG. 2 is a mass spectrum of the arginine glass prepared in Example 1, showing that the resulting amino acid has a stable molecular structure.

FIG. 3 is a degradation curve of the arginine glass prepared in Example 1 in industrial wastewater and industrial soil, proving its degradability.

FIG. 4 is a photograph of a phenylalanine solid glass that is prepared in Example 2 at room temperature and is processable into glass blocks or glass filaments as needed, proving strong processability.

FIG. 5 is microscope photos of the phenylalanine glass prepared in Example 2 and a phenylalanine crystal state in a bright field and a polarized light field, proving that the formed amino acid glass has an amorphous structure.

FIG. 6 is infrared spectrums of the phenylalanine glass prepared in Example 2 and a phenylalanine crystal state, where infrared spectrum peaks of the glass are obviously broadened, showing that molecules in the phenylalanine glass are arranged in disorder.

FIG. 7 is a DSC spectrum and a TG spectrum of a histidine solid glass prepared in Example 3.

FIG. 8 is an XRD pattern of a tyrosine crystal powder and a glass in Example 4.

FIG. 9 is transmittance of an aspartic acid solid glass in Example 5.

FIG. 10 is a sustained release curve of an exenatide glass prepared in Example 5.

FIG. 11 shows release curves of the aspartic acid glass prepared in Example 5 and a crystal.

FIG. 12 is a photograph of a thymopentin glass in Example 8.

FIG. 13 is a glass photograph formed from the thymopentin glass in Example 9 in a film tool.

FIG. 14 is a photograph of an exenatide glass in Example 10.

FIG. 15 is a curve of the aspartic acid glass prepared in Example 5 on blood gluclose control in spontaneous type II diabetic mice.

FIG. 16 is an XRD pattern of a Boc-Val glass in Example 6 and a crystal powder.

FIG. 17 is a polarized light microscope photo of a solid glass of a composition of N-acetyl-L-threonine and L-arginine in Example 7.

FIG. 18 is pharmacodynamic evaluation of a thymopentin solid glass in Example 8 in rats.

FIG. 19 is neurological score of glass Tuftsin in mice with experimental autoimmune encephalomyelitis (EAE) in Example 9.

FIG. 20 is a photograph of a microneedle prepared in Example 29.

FIG. 21 is an optical waveguide photo of laser transmission through a phenylalanine glass fiber optical prepared in Example 31.

FIG. 22 is a process of an analog circuit disappearing when exposed to water in Example 32.

FIG. 23 is a process of an interdigital electrode disappearing when exposed to water in Example 33.

### DETAILED DESCRIPTION

Technical schemes of the present disclosure are described in detail below with reference to examples, which, however, do not impose any limitation on contents claimed in the present disclosure.

### Example 1

A method for preparing an arginine-based bioglass included steps of:
250 ml of 0.01 M aqueous citric acid solution was prepared for later use;
500 mg of L-arginine powder was weighed and placed in a mortar, and uniformly ground;
the arginine powder from the step (2) was placed into a 500 ml round-bottomed flask, and fully dissolved in 20 ml of ultrapure water, to obtain an arginine solution;
20 ml of the aqueous citric acid solution from the step (1) was added to the arginine solution from the step (3), until the arginine solution reached a pH=6.0; and
the arginine solution from the step (4) was heated at a heating rate of 10 K min⁻¹ until the round-bottomed flask reached a temperature of 373 K, and thermostatically treated at this temperature for 2 h to volatilize the solvent. The arginine (Arg)-based glass was obtained.

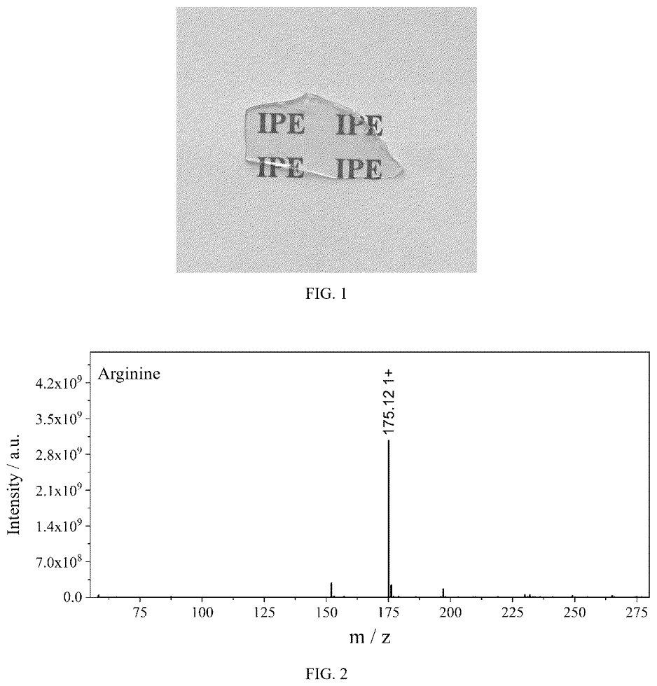

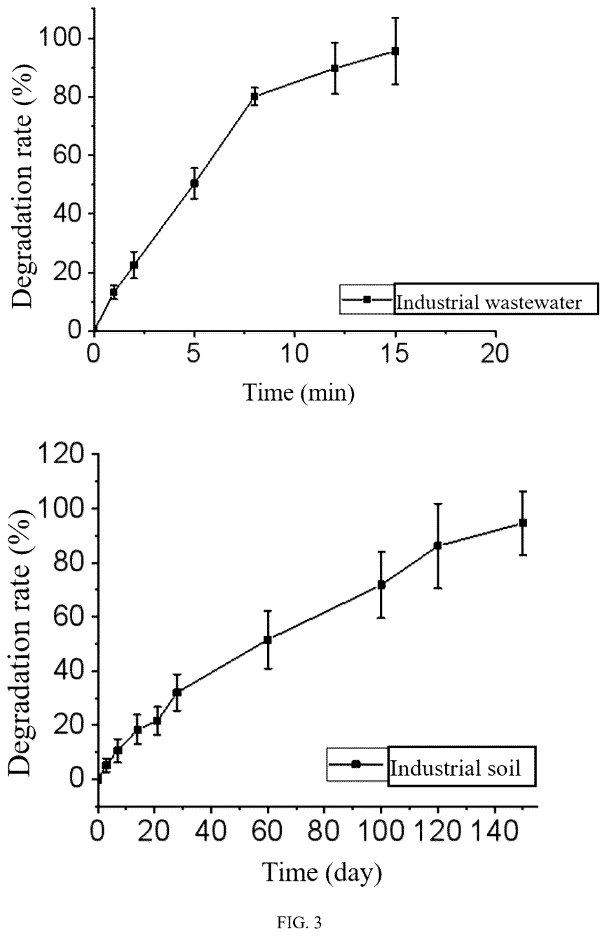

FIG. 1 is a photograph of an arginine glass prepared in Example 1. As shown in FIG. 1, the arginine glass has good optical properties. Its transmittance at 500 nm is further measured using an ultraviolet spectrometer (sample thickness 1 mm), with the result listed in Table 1. Mass spectrum of the resulting arginine glass is shown in FIG. 2. The result shows that the resulting amino acid has a stable molecular structure without occurrence of oxidation or decomposition. The resulting glass has a glass transition temperature of Tg=337 K, a melting point of Tm=495 K, and a Tg/Tm ratio of 0.68, showing that it has a good glass-forming ability. FIG. 3 is a degradation curve of the arginine glass (mass 32.2 mg) prepared in Example 1 in industrial soil and industrial wastewater, proving that the arginine glass is degraded in the environment.

### Example 2

A method for preparing a phenylalanine-based bioglass included steps of:
250 ml of 0.2 M aqueous citric acid solution was prepared for later use;
1 g of L-phenylalanine powder was weighed and placed in a mortar, and uniformly ground;
the phenylalanine powder from the step (2) was placed into a 500 ml round-bottomed flask, and fully dissolved the phenylalanine powder in 20 ml of ultrapure water, to obtain a phenylalanine solution;
20 ml of the aqueous citric acid solution from the step (1) was added to the phenylalanine solution from the step (3), until the phenylalanine solution reached a pH=5.0;
the phenylalanine solution from the step (4) was heated at a heating rate of 40 K min⁻¹ until the round-bottomed flask reached a temperature of 363 K, the temperature was kept at 363 K for 2.5 h; and
the solution was kept at the constant temperature for 30 min after the solution in the round-bottomed flask from the step (5) became viscous, then the device was cooled to 273 K at a cooling rate of 40 K min⁻¹, and kept for 30 min. The phenylalanine (Phe) biomolecular glass was obtained.

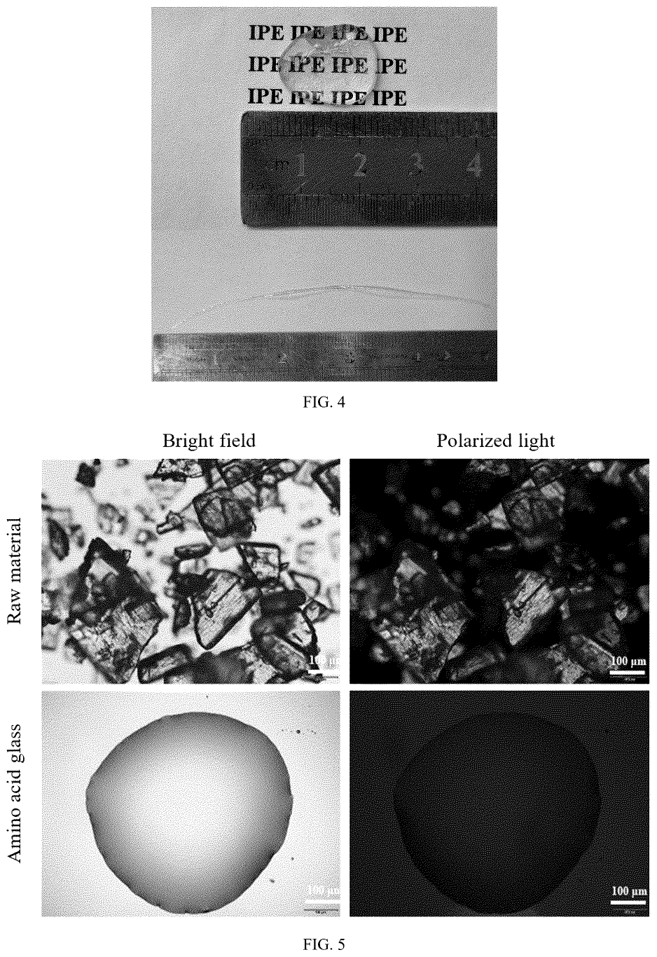

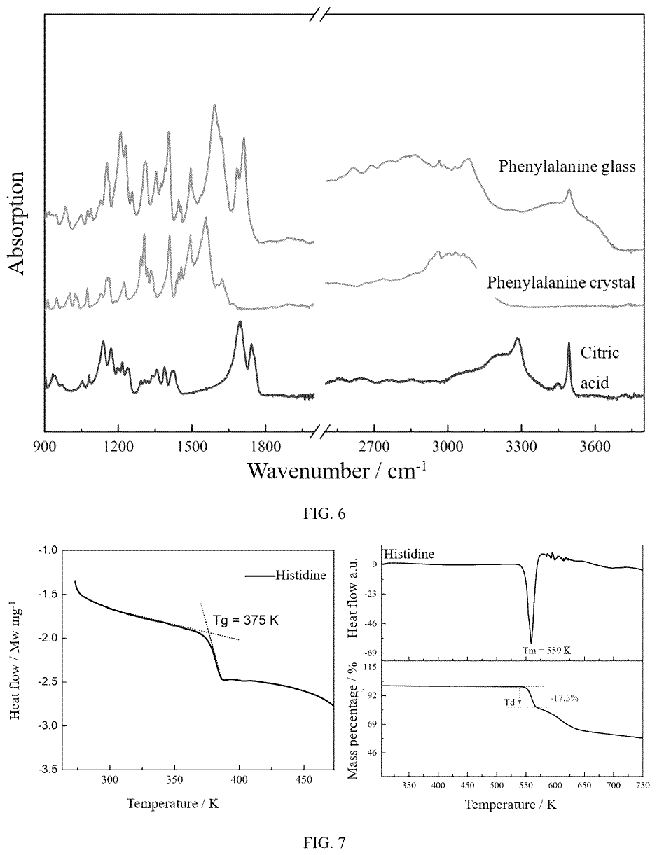

FIG. 4 shows a photograph of the resulting phenylalanine solid glass at room temperature, processable into glass blocks or glass filaments as needed, proving strong processability. FIG. 5 is microscope photos of the phenylalanine glass prepared in Example 2 and a phenylalanine crystal state in a bright field and a polarized light field, proving that the formed amino acid glass has an amorphous structure. FIG. 6 is infrared spectrums of the phenylalanine glass prepared in Example 2 and a phenylalanine crystal state, where infrared spectrum peaks of the glass are obviously broadened, showing that molecules in the phenylalanine glass are arranged in disorder. The resulting glass was further measured to have a glass transition temperature of Tg=400 K, a melting point of Tm=556 K, and a Tg/Tm ratio of 0.72, showing that it has a good glass-forming ability.

### Example 3

A method for preparing a histidine-based bioglass included steps of:
250 ml of 0.1 M aqueous hydrochloric acid solution was prepared for later use;
1 g of L-histidine powder was weighed and placed in a mortar, and uniformly ground;
the histidine powder from the step (2) was placed into a 500 ml round-bottomed flask, and fully dissolved the histidine powder respectively in 20 ml of ultrapure water and 1 ml of ethanol, to obtain a histidine solution;
20 ml of the aqueous hydrochloric acid solution from the step (1) was added to the histidine solution from the step (3), until the histidine solution reached a pH=5.0; and
the histidine solution from the step (4) was heated at a heating rate of 50 K min⁻¹ until the round-bottomed flask reached a temperature of 383 K, the temperature was kept at 383 K for 1.5 h; then the device was cooled to a temperature of 293 K at a cooling rate of 50 K min⁻¹, and kept for 30 min. The histidine (His) solid glass was obtained.

FIG. 7 is a DSC spectrum and a TGA spectrum of the histidine solid glass prepared in Example 3. It has a glass transition temperature of Tg=375 K, a melting point of Tm=559 K, and a Tg/Tm ratio is 0.67, showing that the solid histidine glass has a good glass-forming ability.

### Example 4

A method for preparing a tyrosine-based bioglass included steps of:
100 ml of 0.5 M aqueous acetic acid solution was prepared for later use;
2 g of L-tyrosine powder was weighed and placed in a mortar, and uniformly ground;
the tyrosine powder from the step (2) was placed into a 500 ml round-bottomed flask, and fully dissolved the tyrosine powder in 30 ml of ultrapure water, to obtain a tyrosine solution;
20 ml of the aqueous acetic acid solution from the step (1) was added to the tyrosine solution from the step (3), until the tyrosine solution reached a pH=5.5; and
the tyrosine solution from the step (4) was heated at a heating rate of 50 K min⁻¹ until the round-bottomed flask reached a temperature of 383 K, the temperature was kept at 383 K for 10 min;
the device in the step (5) was cooled to a temperature of 293 K at a cooling rate of 50 K min⁻¹; and
the solvent in the tyrosine solution from the step (6) was volatilized at room temperature, and kept for 20 days. The tyrosine bioglass was obtained.

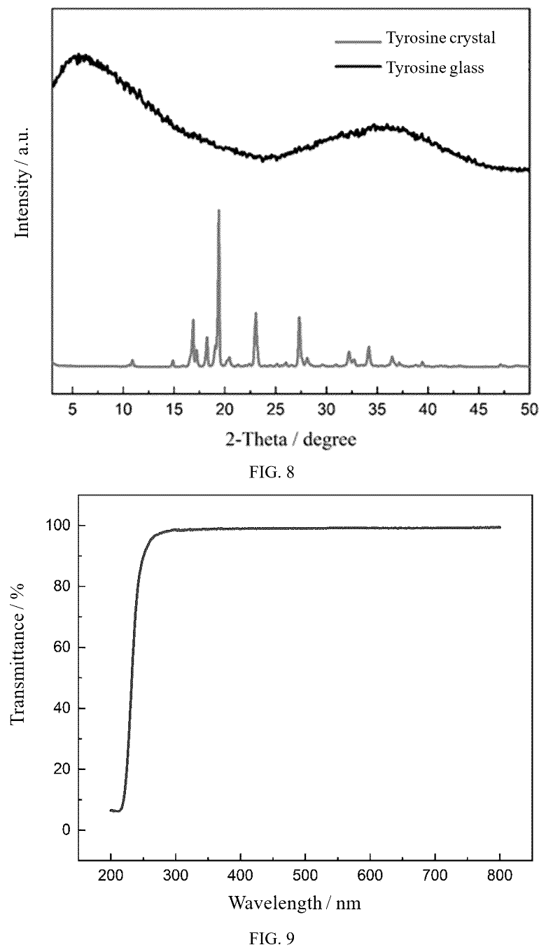

FIG. 8 is an XRD pattern of a tyrosine crystal powder and a glass in Example 4, proving that the tyrosine glass is in an amorphous state. The resulting glass was further measured to have a glass transition temperature of Tg=344 K, a melting point of Tm=617 K, and a Tg/Tm ratio of 0.67, showing that it has a good glass-forming ability.

### Example 5

A method for preparing an aspartic acid-based bioglass included steps of:
500 ml of 0.3 M aqueous citric acid solution was prepared for later use;
500 mg of L-aspartic acid powder was weighed and placed in a mortar, and uniformly ground;
the aspartic acid powder from the step (2) was placed into a 500 ml round-bottomed flask, and fully dissolved the aspartic acid powder in 30 ml of ultrapure water, to obtain an aspartic acid solution;
20 ml of the aqueous citric acid solution from the step (1) was added to the aspartic acid solution from the step (3), until the aspartic acid solution reached a pH=5.0; and
the aspartic acid solution from the step (4) was heated at a heating rate of 30 K min⁻¹ until the round-bottomed flask reached a temperature of 383 K, the temperature was kept at 383 K for 3 h; the solution was cooled to a temperature of 293 K at a cooling rate of 50 K min⁻¹; and kept for 30 min. The aspartic acid (Asp) bioglass was obtained.

FIG. 9 is transmittance of the aspartic acid solid glass in Example 5, approximate to 99%. The resulting glass was further measured to have a glass transition temperature of Tg=377 K, a melting point of Tm=503 K, and a Tg/Tm ratio of 0.75, showing that it has a good glass-forming ability.

### Example 6

A method for preparing a valine-based bioglass included steps of:
500 ml of 0.01 M aqueous malic acid solution was prepared for later use;
500 mg of N-tert-butoxycarbonyl-L-valine (Boc-Val) powder was weighed and placed in a mortar, and uniformly ground;
the Boc-Val powder from the step (2) was placed into a 500 ml round-bottomed flask, and fully dissolved the Boc-Val powder in 30 ml of ultrapure water and 3 ml of glycerol respectively, to obtain a Boc-Val solution;
20 ml of the aqueous malic acid solution from the step (1) was added to the Boc-Val solution from the step (3), until the Boc-Val solution reached a pH=5.5;
the Boc-Val solution from the step (4) was heated to a temperature of 323K at a heating rate of 10 K min⁻¹, the temperature was kept at 323 K for 5 h; and
the N-tert-butoxycarbonyl-L-valine (Boc-Val)-based glass was obtained after the solution in the round-bottomed flask from the step (5) became viscous.

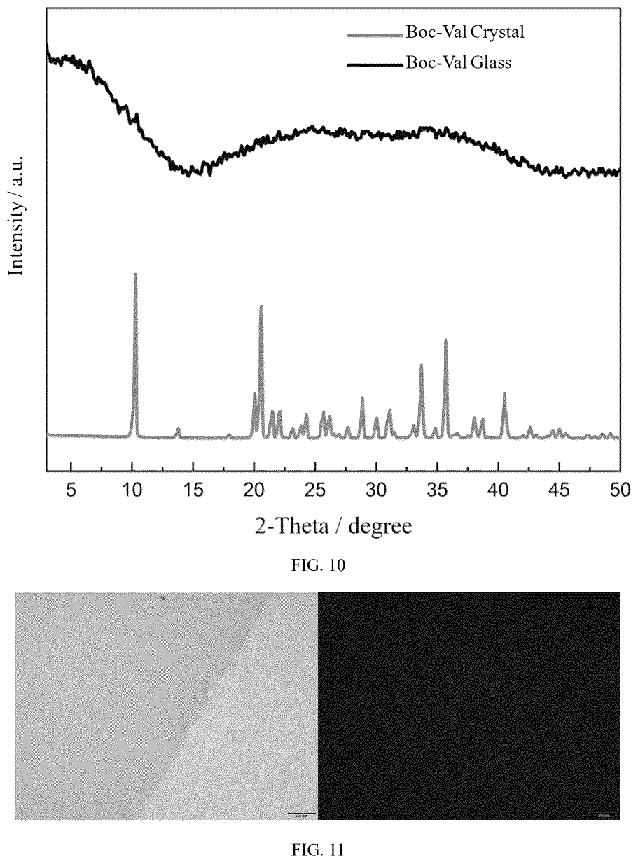

FIG. 10 is an XRD pattern of a Boc-Val glass and a crystal powder in Example 6, proving that the Boc-Val glass is in an amorphous state. The resulting glass was further measured to have a glass transition temperature of Tg=406 K, a melting point of Tm=588 K, and a Tg/Tm ratio of 0.69, showing that it has a good glass-forming ability.

### Example 7

A method for preparing a glass based on a threonine-arginine composition included steps of:
50 ml of 0.01 M aqueous citric acid solution was prepared for later use;
1 g of N-acetyl-L-threonine powder and 1 g of L-arginine powder were weighed and placed in a mortar, and uniformly ground;
the mixed powder of N-acetyl-L-threonine and arginine from the step (2) was placed into a 500 ml round-bottomed flask, and then fully dissolved the mixed powder of the amino acids in 40 ml of ultrapure water, to obtain an amino acid solution;
40 ml of the aqueous citric acid solution from the step (1) was added to the amino acid solution from the step (3), until the amino acid solution reached a pH=5.0; and
the amino acid solution from the step (4) was heated at a heating rate of 50 K min⁻¹ until the round-bottomed flask reached a temperature of 383 K, the temperature was kept at 383 K for 1.5 h; and
the solution was kept at the constant temperature for 20 min after the solution in the round-bottomed flask from the step (5) became viscous, then the device was cooled to a temperature of 293 K at a cooling rate of 50 K min⁻¹; and kept for 30 min. A solid glass of the composition of N-acetyl-L-threonine and L-arginine was obtained.

FIG. 11 is a polarized light microscope photo of the solid glass of the composition of N-acetyl-L-threonine and L-arginine in Example 7 (the left side is a bright field, and the right side is a polarized light field), proving that the mixed solid glass is in an amorphous state.

### Example 8

A method for preparing a thymopentin-based solid glass included steps of:
50 ml of 0.01 M aqueous citric acid solution was prepared for later use;
10 mg of thymopentin powder was weighed and placed in a mortar, and uniformly ground;
the thymopentin powder from the step (2) was placed into a 250 ml round-bottomed flask, and fully dissolved the thymopentin powder in 10 ml of ultrapure water, to obtain a thymopentin solution;
10 ml of the aqueous citric acid solution from the step (1) was added to the amino acid solution from the step (3), until the thymopentin solution reached a pH=6.0; and
the amino acid solution from the step (4) was heated at a heating rate of 50 K min⁻¹ until the round-bottomed flask reached a temperature of 383 K, the temperature was kept at 383 K for 1.5 h; continuing to keep the solution at the constant temperature for 20 min after the solution from the step (5) in the round-bottomed flask became viscous, then the device was cooled to a temperature of 293 K at a cooling rate of 50 K min⁻¹; and kept for 30 min. The thymopentin solid glass was obtained.

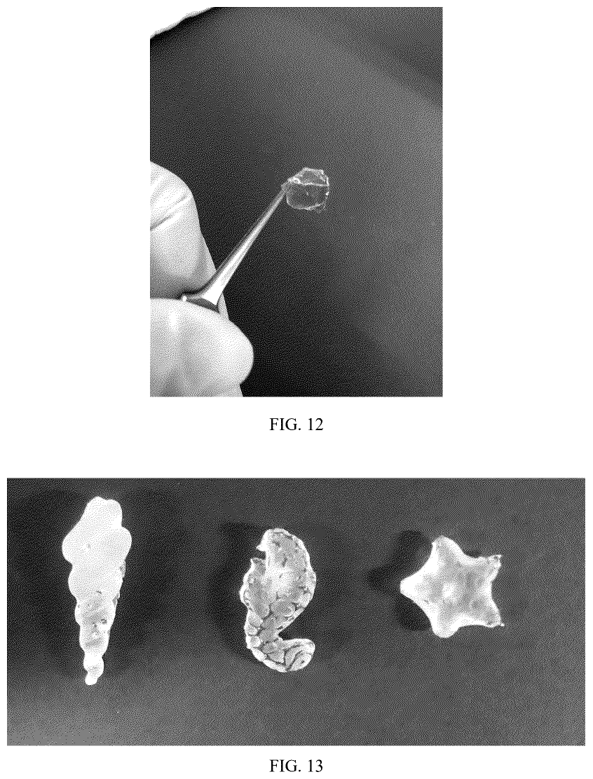

FIG. 12 is a photograph of the transparent thymopentin glass in Example 8.

### Example 9

A method for preparing a tuftsin-based glass included steps of:
50 ml of 0.01 M aqueous malic acid solution was prepared for later use;
10 mg of tuftsin powder was weighed and placed in a mortar, and uniformly ground; the tuftsin powder from the step (2) was placed into a 250 ml round-bottomed flask, and fully dissolved the tuftsin powder in 10 ml of ultrapure water, to obtain a tuftsin solution;
10 ml of the aqueous malic acid solution from the step (1) was added to the amino acid solution from the step (3), until the tuftsin solution reached a pH=6.0; and
the amino acid solution from the step (4) was heated at a heating rate of 10 K min⁻¹ until the round-bottomed flask reached a temperature of 363 K, the temperature was kept at 363 K for 2 h; continuing to keep the solution at the constant temperature for 30 min after the solution from the step (5) in the round-bottomed flask became viscous, then the device was cooled to a temperature of 293 K at a cooling rate of 40 K min⁻¹, and kept for 30 min. The tuftsin solid glass was obtained.

FIG. 13 is a glass formed from the thymopentin glass in Example 9 in a film tool, proving the processability of the peptide glass.

### Example 10

A method for preparing an exenatide-based glass included steps of:
50 ml of 0.01 M aqueous malic acid solution was prepared for later use;
10 mg of exenatide powder was weighed and placed in a mortar, and uniformly ground;
the exenatide powder from the step (2) was placed into a 250 ml round-bottomed flask, and fully dissolved the exenatide powder in 10 ml of ultrapure water, to obtain an exenatide solution;
10 ml of the aqueous malic acid solution from the step (1) was added to the amino acid solution from the step (3), until the exenatide solution reached a pH=6.0; and
the amino acid solution from the step (4) was heated at a heating rate of 10 K min⁻¹ until the round-bottomed flask reached a temperature of 363 K, the temperature was kept at 363 K for 2 h; continuing to keep the solution at the constant temperature for 30 min after the solution from the step (5) in the round-bottomed flask became viscous, then the device was cooled to a temperature of 293 K at a cooling rate of 40 K min⁻¹, and kept for 30 min. The exenatide solid glass was obtained.

FIG. 14 is a photograph of the transparent exenatide glass in Example 10.

### Examples 11-20

The exenatide in Example 10 was replaced with an equal mass of Arg-Arg, Arg-Gly-Asp, Thr-Lys-Pro-Arg, glutathione, vancomycin hydrochloride, Ac-Ala, Ac-Ala-Gln, Cbz-Phe-Phe, Cbz-Phe-Phe-Gly, or Tyr-Ser-Pro-Thr-Ser-Pro-Ser, to prepare a corresponding biomolecular glass, and measure the transmittance and hardness of the resulting glass.

Transmittance measurement method: the glass prepared in each example was processed into a glass wafer with a diameter of 1 cm and a thickness of 1 mm, to measure its transmittance at 500 nm using a UV spectrophotometer (model: Shimadzu UV-1900i).

Hardness measurement method: hardness of the biomolecular glass was measured using a nanoindenter G200 (Agilent Company). Measurement method: the glass material was pressed into a triangular pyramid diamond indenter at a constant strain rate (0.05 s⁻¹), to plot a curve of load force versus displacement to obtain hardness data of each biomolecular glass, with the result as shown in the table below:

| Example No. | Transmittance | Hardness (Mpa) |
|---|---|---|
| Example 11 | 88% | 69 |
| Example 12 | 91% | 101 |
| Example 13 | 83% | 98 |
| Example 14 | 90% | 161 |
| Example 15 | 91% | 195 |
| Example 16 | 92% | 152 |
| Example 17 | 81% | 85 |
| Example 18 | 92% | 275 |
| Example 19 | 86% | 56 |
| Example 20 | 83% | 53 |

### Example 21: Photostability Test of Glass

Specifically, the glass prepared in each example was processed into a glass wafer with a diameter of 1 cm and a thickness of 1 mm, which was transferred in a light environment under lighting conditions of: xenon lamp (Philips), illumination intensity: 6,000 xls; to observe the appearance of the glass wafer on 7 days, 15, and 30 respectively, and measure its transmittance at 500 nm, with the result listed in Table 2.

| | Photostability | | | | | |
|---|---|---|---|---|---|---|
| Example | Day 7 | | Day 15 | | Day 30 | |
| No. | Appearance | Transmittance | Appearance | Transmittance | Appearance | Transmittance |
| Example 1 | Unchanged | 97% | Unchanged | 96% | Unchanged | 95% |
| Example 2 | Unchanged | 98% | Unchanged | 98% | Unchanged | 97% |
| Example 3 | Unchanged | 98% | Unchanged | 97% | Unchanged | 96% |
| Example 4 | Unchanged | 91% | Unchanged | 90% | Unchanged | 90% |
| Example 5 | Unchanged | 98% | Unchanged | 98% | Unchanged | 95% |
| Example 6 | Unchanged | 95% | Unchanged | 94% | Unchanged | 93% |
| Example 7 | Unchanged | 91% | Unchanged | 91% | Unchanged | 91% |
| Example 8 | Unchanged | 92% | Unchanged | 91% | Unchanged | 91% |
| Example 9 | Unchanged | 89% | Unchanged | 88% | Unchanged | 88% |
| Example | Unchanged | 82% | Unchanged | 81% | Unchanged | 80% |
| 10 | | | | | | |

The above result proves that the resulting glass has good photostability performance.

### Example 22: High-Temperature Stability Test of Glass

Specifically, the glass prepared in each example was processed into a glass wafer with a diameter of 1 cm and a thickness of 1 mm, which was transferred in a high-temperature and high-humidity environment (ambient temperature 50° C., 96% relative humidity); to observe the glass on days 0, 15, and 30 respectively, with the result listed in Table 3.

| | High-temperature stability | | | | | |
|---|---|---|---|---|---|---|
| Example | Day 7 | | Day 15 | | Day 30 | |
| No. | Appearance | Transmittance | Appearance | Transmittance | Appearance | Transmittance |
| Example 1 | Unchanged | 98% | Unchanged | 93% | Unchanged | 90% |
| Example 2 | Unchanged | 99% | Unchanged | 96% | Unchanged | 91% |
| Example 3 | Unchanged | 98% | Unchanged | 92% | Unchanged | 90% |
| Example 4 | Unchanged | 92% | Unchanged | 90% | Unchanged | 87% |
| Example 5 | Unchanged | 99% | Unchanged | 94% | Unchanged | 90% |
| Example 6 | Unchanged | 95% | Unchanged | 93% | Unchanged | 92% |
| Example 7 | Unchanged | 91% | Unchanged | 90% | Unchanged | 88% |
| Example 8 | Unchanged | 92% | Unchanged | 90% | Unchanged | 89% |
| Example 9 | Unchanged | 89% | Unchanged | 88% | Unchanged | 87% |
| Example | Unchanged | 82% | Unchanged | 80% | Unchanged | 78% |
| 10 | | | | | | |

The above result proves that the resulting glass has good high-temperature stability performance.

### Example 23: Sustained Release Curve of the Resulting Exenatide Glass

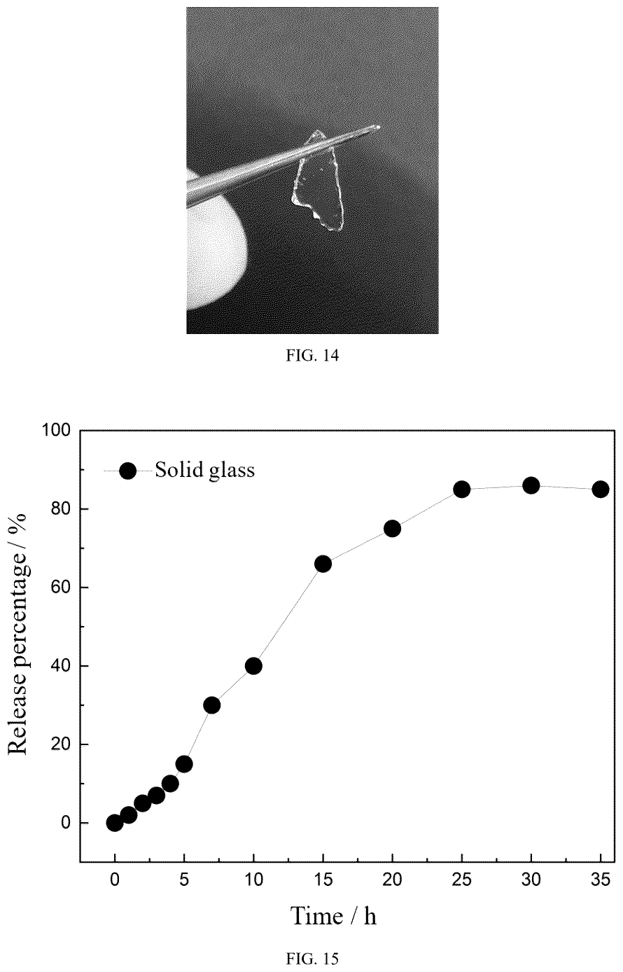

Specifically, the exenatide glass with an initial mass of 30±1 mg and 10 mL of a sustained-release solvent phosphate buffer (0.01 M, pH=6.8) were stirred at a rate of 100 r min⁻¹. At different time points, the supernatant was taken to detect the dissolved-out exenatide using a high-performance liquid chromatograph. High performance liquid chromatography (HPLC) conditions: Liquid phase elution conditions: mobile phase A-water (0.1% TFA), mobile phase B-acetonitrile (0.1% TFA); elution gradient: mobile phase B 20%-60%, 0-16 min; flow rate 1 mL min⁻¹, detection wavelength: 214 nm; the sustained release curve of exenatide was plotted with the result as shown in FIG. 15. The result proves that the exenatide glass can achieve slow release of drugs.

### Example 24: Comparison Between Release Rates of Aspartic Acid Glass and Crystal

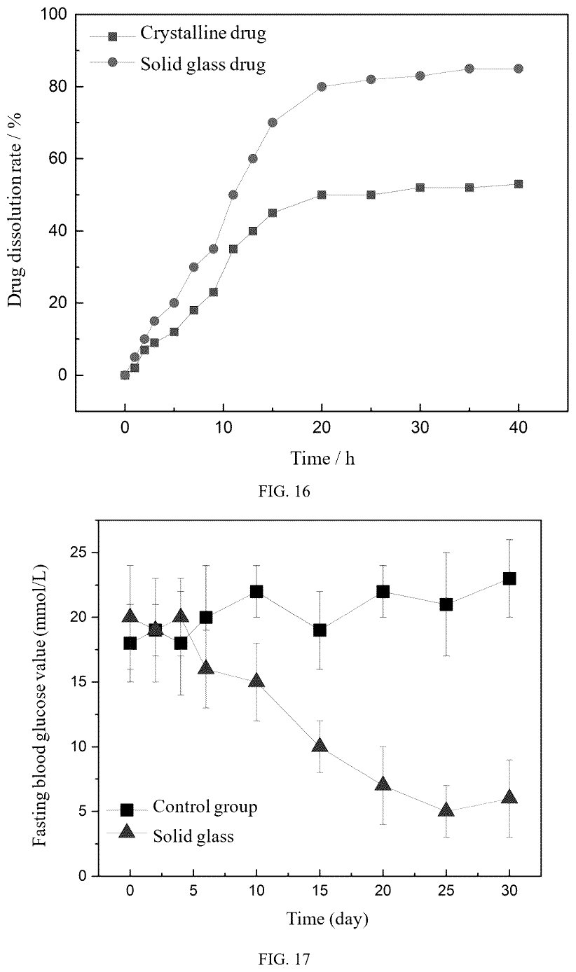

Specifically, the aspartic acid glass with an initial mass of 15±1 mg was mixed with 10 mL of a sustained-release solvent PBS (0.01 M, pH=6.8) at a stirring rate of 100 r min⁻¹, to detect the dissolved-out aspartic acid using a high performance liquid chromatograph under high performance liquid chromatography (HPLC) conditions of: liquid phase elution conditions: mobile phase: methanol and sodium acetate solution (at a volume ratio of 43:57), flow rate: 0.5 mL min⁻¹; column temperature 40° C., detection wavelength: 267 nm; to plot a sustained release curve of the aspartic acid in glassy state. As a control, an equal mass of drug in the amino acid crystal dosage form was weighed, and then incubated in the above sustained-release solution while stirring, to detect the concentration of aspartic acid in the sustained-release solution. FIG. 16 is a release curve of the aspartic acid glass prepared in Example 5 and a crystal, showing that the drug in glassy state can increase the dissolution rate of amino acids.

### Example 25: In Vivo Pharmacodynamic Evaluation of Aspartic Acid Glass

Specifically, purchased spontaneous type II diabetic model mice (male, 8 weeks old) were divided into three groups, with 6 mice in each group. The mice in the experimental group were gavaged with aspartic acid drug in glassy state every day at a dose of 4 mg kg⁻¹, while the mice in the control group were gavaged with an equal mass of normal saline for a test period of 30 days. Tail blood of the mice was taken every day, to measure fasting blood glucose values of the mice with a glucometer. A curve was plotted with time as the abscissa, and the fasting blood glucose values of the experimental mice as the ordinate. FIG. 17 is a curve of the aspartic acid glass prepared in Example 5 on blood glucose control in spontaneous type II diabetic mice, proving that blood gluclose of the mice can be effectively controlled.

### Example 26: Detection of Stability of Exenatide Glass by High Performance Liquid Chromatography (HPLC)

High performance liquid chromatography (HPLC) conditions: liquid phase elution conditions: mobile phase A-water (0.1% TFA), mobile phase B-acetonitrile (0.1% TFA); elution gradient: mobile phase B 20%⁻⁶0%, 0-16 min; flow rate 1 mL min⁻¹, detection wavelength: 214 nm;
the exenatide glass prepared in Example 10 was transferred in a storage chamber maintained at 40° C. and 96% relative humidity (RH) along with an equivalent amount of an exenatide solution. The exenatide content was determined after storage in the chamber for 1, 3, 7, and 14 days. At the designated time points, the array was taken from the chamber and the exenatide glass was dissolved in 0.1 mol L⁻¹ acetic acid (1 mL) to obtain the exenatide solution.

The exenatide content in the resulting washing solution was analyzed by high-performance liquid chromatography (HPLC). The stability of exenatide in the resulting glass was contrastively analyzed with the exenatide solution under same storage conditions as the reference. The percentage of undegraded exenatide remaining in the glass or solution at each time point was determined by measuring the exenatide peak area in the selected sample and dividing it by the initially measured exenatide peak area, with the result listed in Table 2.

| | Exenatide content percentage | | | | |
|---|---|---|---|---|---|
| Sample | Initial | Day 1 | Day 3 | Day 7 | Day 14 |
| Solution control | 100 | 45 | 9 | 1 | 0 |
| Example 9 | 100 | 97 | 93 | 79 | 62 |

The above result shows that the exenatide glass has significantly better stability than the solution group in the 14-day accelerated stability experiment, showing that the resulting exenatide glass can effectively preserve the activity of bioactive molecules and facilitates long-term storage.

### Example 27: Pharmacodynamic Evaluation of Thymopentin Glass in Rats

Experimental method: 28 SPF-grade male SD rats with body weights of 180-220 g without taking any other drugs before the experiment. Thymopentin powder was dissolved in normal saline to prepare a thymopentin solution for use as a reference formulation. The test formulation was the drug in the thymopentin glass dosage form prepared in Example 8. 28 SD rats were randomly divided into 4 groups, named groups 1-4 respectively, with 7 rats in each group, for in vivo pharmacodynamic study.

An immunosuppression model was constructed before starting the experiment. An immunosuppressant cyclophosphamide powder was purchased from Jiangsu Hengrui Pharmaceuticals Co., Ltd and prepared into 1 mg L⁻¹ cyclophosphamide solution with normal saline. Except for the group 1, rats in the other 3 groups were intraperitoneally injected with the immunosuppressant at a dose of 35 mg kg⁻¹ d⁻¹ for three consecutive days to suppress immune functions of the rats. The group 1 served as a blank control group, and was intraperitoneally injected with 35 mL kg⁻¹ of normal saline.

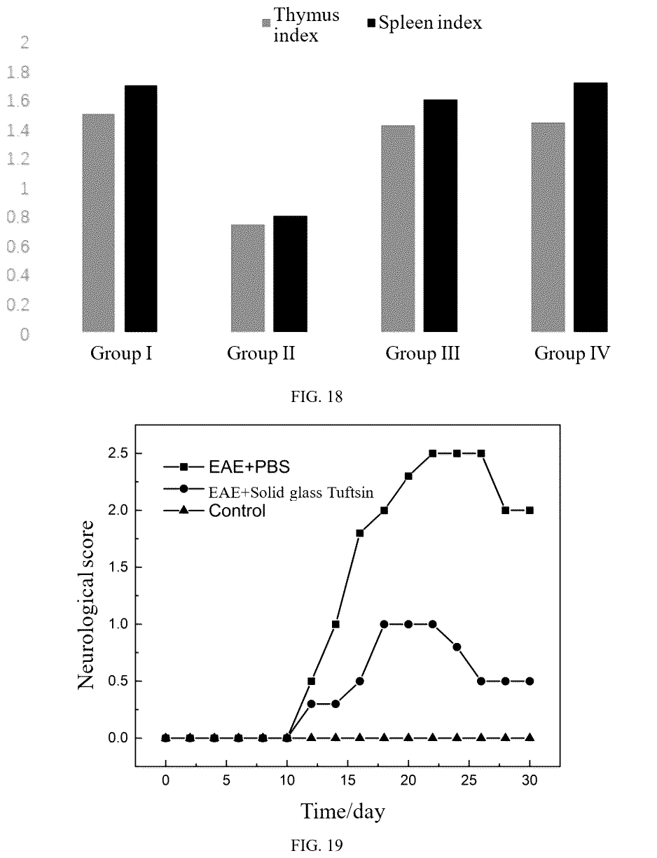

After the model was successfully constructed by injection for three consecutive days, pharmacodynamic evaluation was conducted. The rats in the group 1 served as the blank control group, the rats in the group 2 served as a negative control group, and they were all subcutaneously intravenously injected with 1 mL kg⁻¹ of normal saline for seven consecutive days. The rats in the group 3 served as a positive control group, and were subcutaneously injected with the thymopentin solution at a dose of 100 ug kg⁻¹. The rats in the group 4 were subcutaneously embedded with the thymopentin glass drug prepared in Example 8 at a dose of 100 ug kg⁻¹ for 7 consecutive days. Then, the rats in the 3 groups were killed by cervical dislocation, to collect and weigh the thymus and spleen of each rat, and calculate organ indexes as per the calculation formula below:

$$Organ index = \frac{{W}_{0}}{W}$$

where W₀ is mass of the thymus or spleen, and W is body weight of the rat. As shown in FIG. 18, the negative control group 2 has lower spleen and thymus indexes than the blank control group 1, proving that the immunosuppression model was successfully constructed. Each of the dosing groups 3 and 4 has higher spleen and thymus index values than the negative control group of the model group, the organ indexes of all immunosuppressed rats are improved to some extent, with statistically significant difference, proving that the glass thymopentin has better pharmacodynamical effect than the solution dosage form.

### Example 28: Activity Evaluation of Tuftsin Glass

Experimental model and method: mice with experimental autoimmune encephalomyelitis (EAE) were constructed. Specifically, for the EAE group, 200 μg of MOG35-55 was dissolved in 200 μl of PBS buffer, the mixture was then fully mixed with 200 μl of Freund's complete adjuvant (CFA) to form an emulsion (final concentration of inactivated tuberculin bacillus was 5 mg ml⁻¹), and for the normal control group, 200 μl of PBS was directly fully mixed with 200 μl of CFA. 200 μg of the emulsion was subcutaneously injected at two sites on the back of the mice. 500 ng of pertussis toxin was intraperitoneally injected respectively in 0 h and 48 h after immunization. On day 1 after construction of the EAE model, glass Tuftsin was subcutaneously embedded on the back of the mice at a dose of 100 μg kg⁻¹, for 7 consecutive days, to form a solid glass Tuftsin intervention group.

Neurological scores of experimental mice in each group: After immunization, the experimental mice in each group were evaluated for neurological scores every day and observed continuously for 30 days.

The neurological scores were evaluated based on the following standards: 0 scores mean no symptoms; 1 score means reduced tail strain (complete weakness, tail tip unable to curl) or hindlimb weakness (unsteady gait); 2 scores mean tail and hindlimb weakness; 3 scores mean hindlimb paralysis (unilateral or bilateral hindlimbs can move to a certain extent); 4 scores mean complete hindlimb paralysis (hindlimbs are completely unable to move or are dragged by forelimbs to move); and 5 scores mean death. As indicated by the result shown in FIG. 19, the mice in the solid glass Tuftsin intervention group can effectively restore the nervous systems.

### Example 29: Biomolecular Glass Processed into a Microneedle Device

Based on the casting method of a PDMS mold, the biomolecular glass can be prepared into a microneedle device. Preparation method: The arginine glass prepared in Example 1 was preheated at 80° C., and then poured into a PDMS microneedle female mold, where needle tip and substrate portions of the microneedle mold were filled. After rapid deaeration under reduced pressure under a vacuum pump for 10 min, the mold was cooled to room temperature. After the microneedle body was solidified, the microneedle glass was demolded. A soluble arginine glass microneedle was obtained.

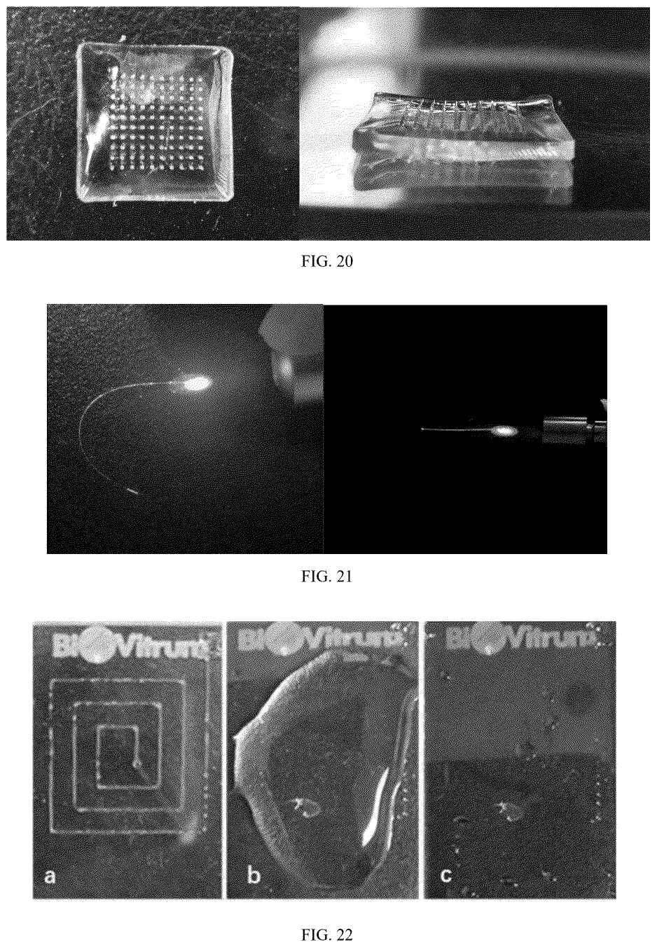

The resulting soluble arginine glass microneedle is as shown in FIG. 20, where the left picture is a top view of the microneedle, and the right picture is a side view of the microneedle, showing that the resulting microneedle body is in order and complete.

### Example 30: Preparation of Rigid Biodegradable Coating from Biomolecular Glass

The viscous vancomycin fluid in the preparation process of Example 14 was coated on a surface of a flexible PEI electrode, which was further heated to obtain a flexible electrode with a vancomycin glass coating. When the electrode is implanted in vivo, the flexible electrode can be endowed with implantation rigidity. When the electrode is implanted in mice, the vancomycin glass coating gradually degrades when being exposed to a physiological solution environment, so that, on the one hand, the electrode returns to a flexible state and maintains its good conformal deformation. The vancomycin glass coating can be used as a sustained-release anti-inflammatory drug to reduce electrode implantation damages.

### Example 31: Biomolecular Glass Processed into an Optical Waveguide Device

The phenylalanine glass prepared in Example 2 was melt-stretched to obtain corresponding glass filaments, which were subjected to optical waveguide testing using a laser device at 532 nm and 616 nm respectively, with the result as shown in FIG. 21. FIG. 21 (left) shows a transmission photo of 532 nm laser, and FIG. 21 (right) shows a transmission photo of 616 nm laser. It can be seen that the laser can be transmitted along the glass filaments, showing that the biomolecular glass can be used in the optical waveguide device.

### Example 32: Biomolecular Glass Processed into Transient Electronic Device

The arginine glass prepared in Example 1 was 3D-printed to form an analog circuit (Panel (a) of FIG. 22). The analog circuit has a machining precision of 0.2 mm. When the analog was exposed to a water environment (trigger conditions) (Panel (b) of FIG. 22), the biomolecular glass was rapidly dissolved, so that the analog circuit pattern was rapidly destroyed, until disappeared (Panel (c) of FIG. 22). This property shows that the biomolecular glass can be used as, e.g., an interconnection wire material in the fabrication of the transient electronic device.

### Example 33: Biomolecular Glass Processed into Transient Electronic Device

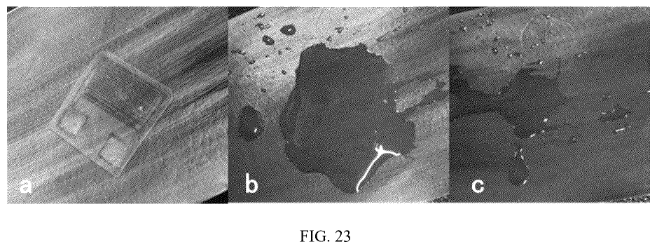

The arginine glass prepared in Example 1 was casted to prepare a transparent substrate film, and then form an interdigital electrode by vacuum evaporation using a stainless-steel mask (Panel (a) of FIG. 23). The interdigital electrode has a machining precision of 0.1 mm. When the interdigital electrode was exposed to a water environment (trigger conditions), the biomolecular glass substrate film can be rapidly dissolved in 10 seconds (Panel (b) of FIG. 23), the interdigital electrode can instantly disappear, and the biomolecular substrate film was also completely dissolved (Panel (c) of FIG. 23). This property shows that the biomolecular glass can be used as, e.g., a substrate material and an encapsulation material in the fabrication of the transient electronic device.

What is claimed is:

1. A biomolecular glass, obtained by volatilizing a solvent in a biomolecular raw material solution, wherein the biomolecular raw material solution comprises a biomolecular raw material, a regulator, and the solvent; the biomolecular raw material comprises a natural amino acid or a peptide, or an amino acid derivative or a peptide derivative, or a mixture of two or more thereof.

2. The biomolecular glass of claim 1, wherein the natura amino acid is one or more selected from the group consisting of glycine, alanine, valine, leucine, isoleucine, methionine, proline, tryptophan, serine, tyrosine, cysteine, phenylalanine, asparagine, glutamine, threonine, aspartic acid, glutamic acid, lysine, arginine, histidine, selenocysteine, pyrrolysine, or a salt thereof,
the peptide is a molecule formed by peptide bond condensation of n of the natural amino acids or a salt thereof, wherein n≥2;
the amino acid derivative is an amino acid derivative of the natural amino acid with a protecting group, wherein the protecting group is one or more selected from the group consisting of Trt, Boc, Fmoc, Cbz/Z, Allyl, C2-C18 acyl, benzoyl, naphthoyl, OFm, Otbu, OBzl, OAll, OMe, and OEt; and
the peptide derivative refers to the peptide with at least one natural amino acid thereof being substituted with the amino acid derivative.

3. The biomolecular glass of claim 1, wherein the regulator is selected from the group consisting of a nucleotide, a nucleotide polymer, RNA, DNA, and other regulator, or a mixture of two or more thereof, the nucleotide is one or more selected from the group consisting of adenosine-5′-monophosphate (AMP), nicotinamide mononucleotide (NMN), guanosine-5′-monophosphate (GMP), uridine-5′-monophosphate (UMP), cytidine-5′-monophosphate (CMP), and adenosine-3′-monophosphate (3-AMP); and the other regulator is one or more selected from the group consisting of D/L-tartaric acid, hydrochloric acid, sulfuric acid, phosphoric acid, lactic acid, polylactic acid, hyaluronic acid, alginic acid, lactobionic acid, citric acid, tartaric acid, oxalic acid, D/L-malic acid, maleic acid, pseudolaric acid, acamprosate, quinic acid, adipic acid, fumaric acid, hexanoic acid, heptanoic acid, octanoic acid, valeric acid, butyric acid, propionic acid, and glacial acetic acid.

4. The biomolecular glass of claim 1, wherein the solvent is water or a salt solution thereof.

5. The biomolecular glass of claim 1, wherein the biomolecular glass is an amorphous body in glassy state, has a light transmittance of 40% to 99%, at a thickness of 1 mm and a wavelength of 500 nm.

6. The biomolecular glass of claim 1, wherein a mass percentage of the biomolecular raw material in the glass is 50% to 100%.

7. The biomolecular glass of claim 1, wherein the biomolecular glass is prepared by a preparation method comprising steps of:
(1) weighing a biomolecular raw material, uniformly mixing and grinding the biomolecular raw material;
(2) dissolving raw material powder from the step (1) in a solvent to obtain a biomolecular solution;
(3) adding a regulator to the biomolecular solution from the step (2), and adjusting a final pH value of the solution by controlling an amount of the regulator; and
(4) transferring an aqueous solution from the step (3) in a container, and volatilizing the solvent by adjusting a temperature of the solution and/or pressurizing the solution, and/or vacuumizing the solution, to prepare the biomolecular glass.

8. The biomolecular glass of claim 7, wherein the final pH is 1-8.

9. An implantable or non-implantable medical material the biomolecular glass of claim 1.

10. The biomolecular glass of claim 1, used in medical care, health monitoring, security encryption, information transfer, and storage.

11. The biomolecular glass of claim 2, wherein 2≤n≤10.

12. The biomolecular glass of claim 1, wherein the biomolecular glass is an amorphous body in glassy state, has a light transmittance of 80% to 99%, at a thickness of 1 mm and a wavelength of 500 nm.

13. The biomolecular glass of claim 1, wherein the biomolecular glass is an amorphous body in glassy state, has a hardness greater than 10 MPa, at a thickness of 1 mm and a wavelength of 500 nm.

14. The biomolecular glass of claim 1, wherein the biomolecular glass is an amorphous body in glassy state, has a hardness greater than 50 MPa, at a thickness of 1 mm and a wavelength of 500 nm.

15. The biomolecular glass of claim 1, wherein a mass percentage of the biomolecular raw material in the glass is 85% to 100%.

16. The biomolecular glass of claim 1, wherein a mass percentage of the regulator in the glass is 0 to 50%.

17. The biomolecular glass of claim 1, wherein a mass percentage of the regulator in the glass is 0 to 15%.

18. The biomolecular glass of claim 7, wherein the final pH is 4-7.

19. The biomolecular glass of claim 1 for piercing skin, mucosa, blood-eye barrier, or blood-brain barrier.

20. The implantable or non-implantable medical material of claim 9, wherein the implantable or non-implantable medical material is a bone stent, a heart stent, and a vascular stent.
